(19) United States
(12) Patent Application Publication
(10) Pub. No.: US 2024/0150208 A1
(43) Pub. Date: May 9, 2024

(54) CONSUMPTION OF AT LEAST ONE COMPOUND FROM A FLUID

(71) Applicant: Industrial Phycology Limited, West Midlands (GB)

(72) Inventors: Thea Ekins-Coward, Somerset (GB); Francesca HO, Bristol (GB); Jeremy Winter, Bristol (GB); Daniel Murray, Somerset (GB)

(21) Appl. No.: 18/284,472

(22) PCT Filed: Mar. 3, 2022
(86) PCT No.: PCT/GB2022/050826

§ 371 Date: Sep. 27, 2023

(30) Foreign Application Priority Data
Mar. 31, 2021 (GB) 2104644.6

(51) Int. Cl. C02F 3/32; C02F 101/10; C02F 101/16

(52) U.S. Cl. CPC C02F 3/322; C02F 2101/105; C02F 2101/16; C02F 2301/08

(57) ABSTRACT

An apparatus and method for consumption of at least one compound from a fluid are disclosed. A method, comprising: providing processed algae for subsequent use in accelerated consumption of at least one compound from a fluid to be treated by exposing algae to a processing fluid depleted in the at least one compound to be consumed by the algae in a processing vessel; adding said processed algae to a contact receptacle containing said fluid to be treated to reduce a concentration of said at least one compound from said fluid to be treated; and recirculating at least a portion of said algae and said processing fluid from said contact receptacle to said processing vessel. This makes the processed algae better adapted to reduce the amount or concentration of the at least one compound within the fluid more quickly than is possible with unprocessed algae.

### FIELD OF THE INVENTION

The present invention relates to an apparatus and method for consumption of at least one compound from a fluid.

### BACKGROUND

Techniques for removing or reducing the concentration of compounds in a fluid are known. For example, metal salt dosing can be used to precipitate compounds into a solid that can be removed with a sedimentation or a filtration process. Anaerobic bacteria can be used to improve phosphorous removal using accumulating bacteria. Reed beds can be utilised to reduce levels of ammonia and reduce biological oxygen demand (BOD), total suspended solids (TSS) and absorb phosphorous from wastewater. High rate algal ponds can use the energy from light to take up nutrients from their environment along with carbon dioxide.

Although all of these techniques can be used to remove compounds from fluids, they each have their own shortcomings. Accordingly, it is desired to provide an improved technique for consumption of at least one compound from a fluid.

### SUMMARY

According to a first aspect, there is provided a method, comprising: providing processed algae for subsequent use in accelerated consumption of at least one compound from a fluid to be treated by exposing algae to a processing fluid depleted in the at least one compound to be consumed by the algae in a processing vessel; adding said processed algae to a contact receptacle containing said fluid to be treated to reduce a concentration of said at least one compound from said fluid to be treated; and recirculating at least a portion of said algae and said processing fluid from said contact receptacle to said processing vessel.

The first aspect recognized that are problems with existing techniques. For example, the use of chemical treatment methods involves hazardous chemicals, tertiary solids removal through filtration, removes dissolved oxygen, requires non-corrosive equipment and may require multi-point dosing. The use of accumulating bacteria is a process which is difficult to keep stable. The use of reed beds requires vast areas of land, bound pollutants can be released into the environment and the plants must be removed periodically. Conventional high-rate algal ponds are shallow with large process footprints and treatment efficiencies are highly dependent on environmental parameters. They are also inefficient in terms of light provision and are dependent on normal metabolic rates of activity for the removal of materials. Also, poor light distribution limits the amount of algae that can be sustained, limiting their efficiency further.

According, a method is provided. The method may comprise providing or producing processed algae. The processed algae may be provided or produced by exposing algae to a processing fluid. The processing fluid may be a fluid which is depleted in the at least one compound to be consumed by algae. The processed algae may be produced in a processing vessel. The processed algae may be provided for subsequent use in accelerated consumption of at least one compound from a fluid. The fluid may be a fluid to be treated. The processed algae may be added to a separate contact receptacle. The contact receptacle may contain the fluid to be treated. The processed algae when exposed to the fluid to be treated may reduce a concentration or amount of at least one compound in that fluid. At least some of the processed algae and/or the processing fluid may be recirculated back to the separate processing vessel for subsequent production of processed algae. In this way, processed algae are provided by exposing, subjecting or suspending algae in a fluid which is typically depleted in the at least one compound to be consumed by the algae. In some embodiments, the processing fluid has previously been in contact with algae although it will be appreciated that other techniques can be used to produce the processing fluid depleted in one or more compounds. The exposure of the algae to the processing fluid conditions the algae by increasing the rate at which the processed algae can consume the at least one compound from the fluid to be treated. This makes the processed algae better adapted to reduce the amount or concentration of the at least one compound within the fluid to be treated more quickly than is possible with unprocessed algae. In some embodiments, the processing vessel used to produce the processed algae is arranged in and operates in parallel with a contact vessel which uses the processed algae. That arrangement allows the production and use of the processed algae to occur in parallel. In some embodiments, the processing vessel provides the appropriate condition (the presence of the processing fluid) and a selected residence time of the algae within the processing vessel to change the algae's metabolism.

The processing fluid may comprise an algae-contacted fluid which has been in prior contact with algae.

The processing fluid or algae-contacted fluid may be depleted in at least one compound to be consumed by the algae.

The exposing the algae to the processing fluid or algae-contacted fluid may increase a capacity of the processed algae to consume the at least one compound in the fluid compared to the algae prior to processing.

The exposing the algae to the processing fluid or algae-contacted fluid may up-regulate at least one of a biological, physical and chemical mechanism to consume the at least one target compound in the processed algae compared to the algae prior to processing. Typically, the exposing will occur for a selected residence time. Such exposing occurs in a separate vessel to a contact receptacle.

The exposing the algae to the processing fluid or algae-contacted fluid may up-regulate at least one of cell membrane biological, diffusion and disassociation kinetic transporters of the at least one compound in the processed algae compared to the algae prior to processing.

The exposing the algae to the processing fluid or algae-contacted fluid may occur in conditions which promote a reduction of the at least one compound retained by the processed algae compared to the algae prior to processing.

The method may comprise recirculating at least a portion of said processed algae by adding said least said portion of said processed algae to said fluid.

The processed algae may comprise conditioned algae. The exposing the algae to the processing fluid or algae-contacted fluid may occur in conditions which promote an up to 10 times increase in rate of reduction of the at least one compound retained by the conditioned algae compared to the algae prior to conditioning.

The exposing the algae to the processing fluid or algae-contacted fluid may occur in conditions which promote a depletion of the at least one compound retained by the conditioned algae.

The exposing the algae to the processing fluid or algae-contacted fluid may occur in conditions which promote a depletion of the at least one compound retained or internalised by the conditioned algae to less than an amount of that compound when the algae is in a normal metabolic state.

The exposing the algae to the processing fluid or algae-contacted fluid may occur in conditions which promote a depletion of the at least one compound retained by the conditioned algae at a faster rate compared to the algae prior to being received by the conditioning vessel.

The exposing the algae to the processing fluid or algae-contacted fluid may occur in conditions which promote an increase in biomass of the conditioned algae compared to the algae prior to conditioning.

The exposing the algae to the processing fluid or algae-contacted fluid may occur in conditions which promote an up to 5 times increase in biomass of the conditioned algae compared to the algae prior to conditioning.

The exposing the algae to the processing fluid or algae-contacted fluid may occur within an illuminated environment.

The exposing the algae to the processing fluid or algae-contacted fluid may comprise diluting pre-conditioned algae with the processing fluid or algae-contacted fluid.

The diluting may comprise utilising up to around 99% by volume of the processing fluid or algae-contacted fluid to dilute the pre-conditioned algae.

The method may comprise recirculating at least a portion of the conditioned algae by adding the least the portion of the conditioned algae to the fluid.

The processed algae may comprise pre-conditioned algae. The method may comprise pre-conditioning the algae by storing the algae to produce the pre-conditioned algae.

An optical density of the fluid-exposed algae when extracted from the processing fluid or algae-contacted fluid may be higher than when in the fluid.

The storing the algae may occur in conditions which promote a reduction of the at least one compound retained by the pre-conditioned algae compared to the algae prior to pre-conditioning.

The storing the algae may occur in conditions which promote an up to 3 times reduction of the at least one compound retained by the pre-conditioned algae compared to the algae prior to pre-conditioning.

The storing the algae may occur in conditions which promote an increase in biomass of the pre-conditioned algae compared to the algae prior to pre-conditioning.

The storing the algae may occur in conditions which promote an up to 3 times increase in biomass of the pre-conditioned algae compared to the algae prior to pre-conditioning.

The storing may comprise storing the algae for a storage period which is no longer than when a reduction in biomass occurs.

The storing the algae may occur for up to 6 days.

The storing may occur in an unilluminated environment.

The storing may occur in a dark environment.

The storing may occur in an agitating environment.

The method may comprise supplying at least a portion of the pre-conditioned algae to the conditioning vessel.

The method may comprise recirculating at least a portion of the pre-conditioned algae by adding at least the portion of the pre-conditioned algae to the fluid.

The method may comprise adding the processed algae, the pre-conditioned algae and/or the conditioned algae to the fluid to consume the at least one compound from the fluid.

The processed algae, the pre-conditioned algae and/or the conditioned algae may consume the at least one compound from the fluid at a rate which is faster than algae prior to the processing, pre-conditioning and/or conditioning.

The adding may comprise adding at least a recirculated portion of the processed algae, the pre-conditioned algae and/or the pre-conditioned algae to the fluid.

The adding may comprise adding the pre-conditioned algae and the conditioned algae in a ratio of up to 90% by volume of the pre-conditioned algae with the remainder being the conditioned algae.

The adding may comprise adding the pre-conditioned algae to the fluid prior to adding the conditioned algae.

The adding may comprise adding the pre-conditioned algae to reduce an amount of the at least one compound in the fluid prior to adding the conditioned algae.

The adding may comprise adding the conditioned algae to further reduce an amount of the at least one compound in the fluid.

The method may comprise storing the fluid for up to 24 hours with the pre-conditioned algae and the conditioned algae.

At least one of the processed algae, the conditioned algae and the pre-conditioned algae may consume an increased amount of the at least one compound from the fluid compared to algae prior to at least one of processing, conditioning and pre-conditioning.

At least one of the processed algae, the conditioned algae and the pre-conditioned algae may consume up to 80 times increase in amount of the at least one compound from the fluid compared to algae prior to at least one of processing, conditioning and pre-conditioning.

At least one of the processed algae, the conditioned algae and the pre-conditioned algae may consume up to 80 times increase in amount of the at least one compound from the fluid compared to compared to a metabolic amount of the at least one compound consumed by algae prior to at least one of processing, conditioning and pre-conditioning.

The compound may comprise at least one of a phosphate, nitrogenous, pharmaceutical compounds, metabolites and metallic elements.

The method may comprise producing the processing fluid or algae-contacted fluid by initially adding starter algae to the fluid and supporting normal metabolic removal of the at least one compound.

The supporting normal metabolic removal of the at least one compound may occur through at least one of a biological, physical and chemical mechanism.

The supporting normal metabolic removal of the at least one compound may occur using low affinity cell membrane transporters.

The supporting normal metabolic removal of the at least one compound may occur for up to fourteen days.

The algae may at least partially comprise fluid-exposed algae extracted from the algae-contacted fluid.

The method may comprise harvesting excess fluid-exposed algae from the processing fluid or algae-contacted fluid.

The processing fluid or algae-contacted fluid may at least partially comprise fluid extracted following contact with the algae.

The fluid-exposed algae and the processing fluid or algae-contacted fluid may be extracted by separating at least a portion of the processing fluid or algae-contacted fluid.

According to a second aspect, there is provided a method, comprising: providing processed algae for subsequent use in accelerated consumption of at least one compound from a fluid by exposing algae to an algae-contacted fluid.

According to a third aspect, there is provided a method, comprising: adding algae to a contact receptacle containing a fluid to be treated to reduce a concentration of at least one compound from said fluid to be treated to produce a processing fluid depleted in said at least one compound; extracting at least a portion of said processing fluid from said contact receptacle; providing processed algae for subsequent use in accelerated consumption of at least one compound from a fluid to be treated by exposing algae to an said processing fluid in a processing vessel; and recirculating said processed algae into contact receptacle.

The second and third aspects may have the optional features of the first aspect set out above.

According to a fourth aspect, there is provided an apparatus, comprising: a processing vessel configured to provide processed algae for subsequent use in accelerated consumption of at least one compound from a fluid to be treated by exposing algae to a processing fluid depleted in said at least one compound to be consumed by the algae; a contact receptacle configured to add said processed algae to said fluid to be treated to reduce a concentration of said at least one compound from said fluid to be treated; and a recirculating mechanism configured to recirculate at least a portion of said algae and said processing fluid from said contact receptacle to said processing vessel.

The processing fluid may comprise an algae-contacted fluid may which has been in prior contact with algae. Hence, the algae-contacted fluid may be reused to help condition the algae to adapt the algae for accelerated consumption of the at least one compound from the fluid.

The processing fluid or algae-contacted fluid may be depleted in at least one compound to be consumed by the algae. The processing vessel may be configured to expose the algae to the processing fluid or algae-contacted fluid to increase a capacity or speed of the processed algae to consume the at least compound in the fluid compared to the algae prior to being received by the processing vessel. Accordingly, the processing fluid or algae-contacted fluid may have reduced amounts of the compound to be consumed compared to the amounts of that compound in the fluid to be treated. Providing the processing fluid or algae-contacted fluid with the reduced amount of compound to the processing vessel may cause the algae to adapt to consume that compound more quickly than it would otherwise do so when the algae then comes into contact with that compound subsequently within the fluid to be treated.

The processing vessel may be configured to expose the algae to the processing fluid or algae-contacted fluid to up-regulate or increase the rate of at least one of a biological, physical and chemical mechanism in the processed algae to consume the at least one compound compared to algae prior to being received by the processing vessel. In other words, the consumption mechanism of the algae is increased within the processing vessel. This adapts the processed algae to consume the at least one compound in the fluid faster than it could otherwise. It will be appreciated that this increase in the consumption mechanism is not instantaneous and the processing vessel is configured or operated to provide the required residence time for the cell to respond to the supplied environment within the processing vessel. In some embodiments, there is provided at least one (typically more) processing vessel which runs in parallel to a wastewater treatment or contact vessel.

The processing vessel may be configured to expose the algae to the processing fluid or algae-contacted fluid to up-regulate or increase at least one of a cell membrane biological, diffusion and disassociation kinetic transporter of the at least one compound in the processed algae compared to the algae prior to being received by the processing vessel.

The processing vessel may be configured to expose the algae to the processing fluid or algae-contacted fluid in conditions which promote or enhance a reduction of the at least one compound retained by the processed algae compared to the algae prior to being received by the processing vessel.

The apparatus may comprise a processed algae conduit coupled with an outlet of said processing vessel configured recirculate at least a portion of said processed algae by adding said least said portion of said processed algae to said fluid. In some embodiments, the recirculated algae and the processing fluid or algae-contacted fluid are sent to different locations in the apparatus. For example, recirculated processed algae are provided to the contact receptacle while recirculated processing fluid or algae-contacted fluid is sent to the processing vessel.

The processing vessel may comprise a conditioning vessel configured to provide conditioned algae as the processed algae. The conditioning vessel may be configured to expose the algae to the processing fluid or algae-contacted fluid in conditions which promote an up to 10-times increase in rate of reduction of the at least one compound retained by the conditioned algae compared to the algae prior to being received by the conditioning vessel. This helps to adapt the algae to increase the rate at which the conditioned algae consume the at least one compound during subsequent exposure to a fluid containing that compound.

The conditioning vessel may be configured to expose the algae to the processing fluid or algae-contacted fluid in conditions which promote or enhance a depletion of the at least one compound retained by the conditioned algae. This results in fundamental changes in the cell which is not possible in some existing techniques where the fluid is recirculated to the inlet of the contact receptacle which would, depending on the period of time, just dilute the inlet fluid stream.

The conditioning vessel may be configured to expose the algae to the processing fluid or algae-contacted fluid in conditions which promote or enhance a depletion of the at least one compound retained or internalised by the conditioned algae to less than an amount of that compound when the algae is in a normal metabolic state. It will be appreciated that the conditioning vessel may be configured to expose the algae for a selected residence time in order to achieve this.

The depletion may be of the at least one compound retained within the cell of the conditioned algae. Reducing the amount of the compound to less compared to that amount when the algae is in a normal healthy metabolic state helps to adapt the algae to increase the rate at which the conditioned algae consume the at least one compound during subsequent exposure to a fluid containing that compound.

The conditioning vessel may be configured to expose the algae to the processing fluid or algae-contacted fluid in conditions which promote or enhance a depletion of the at least one compound retained by the conditioned algae at a faster rate compared to the algae prior to being received by the conditioning vessel.

The conditioning vessel may be configured to expose the algae to the processing fluid or algae-contacted fluid in conditions which promote an increase in biomass of the conditioned algae compared to the algae prior to being received by the conditioning vessel. Allowing the algae to grow and increase biomass in the conditioning vessel helps to deplete or reduce the amount of the at least one compound present which further enhances the ability of the conditioned algae to subsequently consume the at least one compound during exposure to the fluid.

The conditioning vessel may be configured to expose the algae to the processing fluid or algae-contacted fluid in conditions which promote or provide an up to five-times increase in biomass of the conditioned algae compared to the algae prior to being received by the conditioning vessel. In other words, typically up to five times the amount of conditioned algae exits the conditioning vessel compared to the amount of algae entering the conditioning vessel.

The conditioning vessel may be configured to expose or subject the algae to the processing fluid or algae-contacted fluid within an illuminated environment. The environment may be naturally or artificially illuminated. The illumination may be submerged.

The conditioning vessel may be configured to expose the algae to the processing fluid or algae-contacted fluid by diluting pre-conditioned algae with the processing fluid or algae-contacted fluid. Hence, the algae provided to the conditioning vessel may already be pre-conditioned.

The conditioning vessel may be configured to utilise up to around 99% by volume of the processing fluid or algae-contacted fluid to dilute the pre-conditioned algae. Hence, a proportion of the processing fluid or algae-contacted fluid may be recycled and is applied to the conditioning vessel.

The apparatus may comprise a conditioned algae conduit coupled with an outlet of the conditioning vessel. The conditioned algae conduit may be configured to recirculate at least a portion of the conditioned algae by adding at least the portion of the conditioned algae to the fluid. Typically, all the conditioned algae may be added to the fluid.

The processed algae may comprise pre-conditioned algae and the processing vessel may comprise a pre-conditioning vessel configured to pre-condition the algae by storing the algae to produce the pre-conditioned algae. Hence, a pre-conditioning state may be provided which takes algae and performs pre-conditioning on that algae. Additional materials may be added into the pre-conditioning vessel to promote the activity and/or depletion of materials or growth of the algae. Steps can also be taken to decrease and/or remove and/or kill-off unwanted organisms if necessary while preserving the viability of the algae. Typically, the pre-conditioned algae is very dense and is provided as a separate stream from the pre-conditioning vessel which runs in parallel with the contact receptacle.

An optical density of the fluid-exposed algae when extracted from the processing fluid or algae-contacted fluid may be higher than when in the fluid.

The pre-conditioning vessel may be configured to store or retain the algae in or under conditions which promote or enhance a reduction or decrease of the at least one compound retained by the pre-conditioned algae compared to the algae prior to pre-conditioning.

The pre-conditioning vessel may be configured to store the algae in conditions which promote an up to three-times reduction of the at least one compound retained by the pre-conditioned algae compared to the algae prior to being received by the pre-conditioning vessel. Hence, the pre-conditioning helps to perform an initial depletion of the at least one compound being retained by the pre-conditioned algae prior to conditioning.

The pre-conditioning vessel may be configured to store the algae in conditions which promote an increase in biomass of the pre-conditioned algae compared to the algae prior to pre-conditioning.

The pre-conditioning vessel may be configured to store the algae in conditions which promote an up to three-times increase in biomass of the pre-conditioned algae compared to the algae prior to pre-conditioning.

The pre-conditioning vessel may be configured to store the algae for a storage period which is no longer than when a reduction in biomass occurs. Accordingly, the pre-conditioning vessel may store the algae until a maximum increase in biomass occurs and prior to an increased mortality of the algae due to insufficient amounts of the at least one compound. Typically, such increase in biomass is achieved independent of the presence of light.

The pre-conditioning vessel may be configured to store the algae for up to six days. This storage residence time relates to the pre-conditioning of the algae rather than treatment of the fluid in the contact receptacle.

The pre-conditioning vessel may be configured to store the algae in an unilluminated environment. By storing the algae in an unilluminated environment, the energy consumption of the pre-conditioning vessel is reduced.

The pre-conditioning vessel may be configured to store the algae in a dark environment.

The pre-conditioning vessel may be configured to store the algae in an agitating or circulating environment. Various techniques for agitating the algae can be used, such as, for example, an aerator.

The pre-conditioning vessel may comprise a pre-conditioned algae conduit which is coupled with an outlet of the pre-conditioning vessel. The pre-conditioned algae conduit may be configured to supply at least a portion of the pre-conditioned algae to the conditioning vessel.

The pre-conditioned algae conduit may be configured to recirculate at least a portion of the pre-conditioned algae by adding at least the portion of the pre-conditioned algae to the fluid. Typically, some of the pre-conditioned algae is added to the fluid, with the balance being provided to the conditioning vessel.

The contact receptacle may be configured to add the processed algae, the pre-conditioned algae and/or the conditioned algae to the fluid to consume or reduce a concentration of the at least one compound from the fluid. Hence, rather than harvesting the algae for specific products such as fertiliser, the apparatus typically recirculates the processed algae for use in the contact receptacle. Also, rather than pre-treating or adapting the fluid entering the apparatus, the apparatus typically pre-treats or processes the algae.

The processed algae, the pre-conditioned algae and/or the conditioned algae may be arranged or adapted to consume the at least one compound from the fluid at a rate which is faster than algae prior to the processing, pre-conditioning and/or conditioning.

The contact receptacle may be configured to add at least a recirculated portion of the processed algae, the pre-conditioned algae and/or conditioned algae to the fluid. Hence, the processed algae, the pre-conditioned algae and/or pre-conditioned algae may be reused within the contact receptacle.

The contact receptacle may be configured to add the pre-conditioned algae and the conditioned algae in a ratio of up to 90% by volume of the pre-conditioned algae, with the remainder being the conditioned algae. This again helps to reduce the energy consumption of the apparatus since the bulk of the algae used to contact the fluid is pre-conditioned algae with the performance being enhanced with conditioned algae. Typically, the contact receptacle commonly utilises overall around 10-30% by volume of conditioned algae (depending on density) and around 0.4-2% of preconditioned (as it is much more concentrated/dense).

The contact receptacle may be configured to add the pre-conditioned algae to the fluid prior to adding the conditioned algae. Typically, the pre-conditioned algae are added and a selected period of time elapses prior to conditioned algae being added so the conditioned algae does not out compete with the pre-conditioned algae which improves efficiency.

The contact receptacle may be configured to add the pre-conditioned algae to reduce an amount of the at least one compound in the fluid prior to adding the conditioned algae. Hence, the pre-conditioned algae may perform an initial reduction in the amount of the compound in the fluid.

The contact receptacle may be configured to add the conditioned algae to further reduce an amount of the at least one compound in the fluid. Hence, the conditioned algae may help to reduce the amount of the compound in the fluid further.

The contact receptacle may be configured to store the fluid for up to 24 hours with the pre-conditioned algae and the conditioned algae. Hence, rather than having a residence time of 5-6 days as in most approaches, the concentration of the at least one compound is reduced to the required amount in less than around 24 hours.

At least one of the processed algae, the conditioned algae and the pre-conditioned algae may be adapted to consume an increased amount of the at least one compound from the fluid compared to algae prior to at least one of processing, conditioning and pre-conditioning. Such algae prior to processing, pre-conditioning and/or conditioning may be algae in a normal metabolic state. This differs to some existing approaches which cannot manipulate the algae for accelerated consumption.

At least one of the processed algae, the conditioned algae and the pre-conditioned algae may be adapted to consume up to an 80-times increase in amount of the at least one compound from the fluid compared to algae prior to at least one of processing, conditioning and pre-conditioning.

At least one of the processed algae, the conditioned algae and the pre-conditioned algae may be adapted to consume up to an 80-times increase in amount of the at least one compound from the fluid compared to a metabolic amount of the at least one compound consumed by algae prior to at least one of processing, conditioning and pre-conditioning.

The compound may comprise at least one of a phosphate, nitrogenous, pharmaceutical compounds, metabolites and metallic elements.

The contact receptacle may be configured to produce the algae-contacted fluid by initially adding starter algae to the fluid and supporting normal metabolic removal of the at least one compound. Accordingly, the contact receptacle (or alternatively even the processing vessel, the pre-conditioning vessel or the conditioning vessel) may be initially provided with unconditioned or unadapted algae, but the these may need to be run at a lower rate or for a longer period of time since the removal or reduction of the at least one compound will occur at a lower rate than with the processed, pre-conditioned or conditioned algae. The normal metabolic removal may comprise removal or reduction of the at least one compound at a normal metabolic rate.

The contact receptacle may be configured to support normal metabolic removal or reduction of the at least one compound using at least one of a biological, physical and chemical mechanism.

The contact receptacle may be configured to support normal metabolic removal or reduction of the at least one compound using low affinity cell membrane transporters. This may occur due to an increased binding to the cell surface or extracellular organic matter (EOM).

The contact receptacle may be configured to support normal metabolic removal or reduction of the at least one compound for up to fourteen days.

The apparatus may comprise an extractor configured to extract fluid-exposed algae from the algae-contacted fluid and to provide the fluid-exposed algae at least partially as the algae. In other words, the extractor may extract fluid-exposed algae and provide that fluid-exposed algae to the processing and/or conditioning vessel (optionally via the pre-conditioning vessel).

An optical density of the fluid-exposed algae when extracted from the processing fluid or algae-contacted fluid may be higher than when in the fluid.

The apparatus may comprise a harvesting receptacle configured to retain excess fluid-exposed algae. In other words, any algae which is not recirculated to the processing vessel or the conditioning vessel (optionally via the pre-conditioning vessel) may be harvested for other uses.

The extractor may be configured to extract fluid following contact with the algae and to provide the extracted fluid at least partially as the processing fluid or algae-contacted fluid. In other words, the algae-contacted fluid may be extracted using the extractor.

The extractor may be configured to separate at least a portion of the processing fluid or algae-contacted fluid to provide the fluid-exposed algae and the algae-contacted fluid.

According to a fifth aspect, there is provided an apparatus, comprising: a processing vessel configured to provide processed algae for subsequent use in accelerated consumption of at least one compound from a fluid by exposing algae to an algae-contacted fluid.

According to a sixth aspect, there is provided an apparatus, comprising: a contact receptacle configured to add algae to a fluid to be treated to reduce a concentration of at least one compound from said fluid to be treated to produce a processing fluid; an extractor configured to extract at least a portion of said processing fluid from said contact receptacle; a processing vessel configure to provide processed algae for subsequent use in accelerated consumption of at least one compound from the fluid to be treated by exposing algae to said processing fluid; and a recirculating mechanism configured to recirculate said processed algae into contact receptacle.

The fifth and sixth aspects may have the optional features of the fourth aspect set out above.

Further particular and preferred aspects are set out in the accompanying independent and dependent claims. Features of the dependent claims may be combined with features of the independent claims as appropriate, and in combinations other than those explicitly set out in the claims.

Where an apparatus feature is described as being operable to provide a function, it will be appreciated that this includes an apparatus feature which provides that function or which is adapted or configured to provide that function.

### BRIEF DESCRIPTION OF THE DRAWINGS

Embodiments of the present invention will now be described further, with reference to the accompanying drawings, in which:

FIG. 1 illustrates schematically an apparatus according to one embodiment;

FIG. 2 shows changes in optical density (indicating the change in biomass concentration) and changes in phosphate retained by fluid-exposed algae whilst within a pre-conditioning vessel;

FIG. 3 shows changes in optical density (indicating change in biomass concentration) and changes in phosphate retained by pre-conditioned algae whilst within a conditioning vessel;

FIG. 4 shows test result phosphate levels from a demonstration facility using batch processing of 25,000 litres of fluid 10 over a two-week period;

FIG. 5 shows test result phosphate and total nitrogen levels from a demonstration facility using batch processing;

FIG. 6 shows test result water phosphate reduction rates and algae phosphate increase rates in conditioned algae; and

FIG. 7 shows test result water phosphate reduction rates using either solely pre-conditioned algae or solely conditioned algae.

### DESCRIPTION OF THE EMBODIMENTS

Before discussing embodiments in any more detail, first an overview will be provided. Some embodiments provide an arrangement where the properties of algae are optimized or adapted to take up, consume or reduce specific compound(s) such as phosphates, nitrogenous, heavy metals, pharmaceuticals, organic compounds, as well as reducing pathogens present within a fluid such as commercial, industrial or domestic wastewater or aquatic medium. Typically, the algae are exposed in a separate processing vessel for a selected period to a fluid (such as a processing fluid) which has depleted concentrations of one or more compounds which are desired to be consumed by the algae. Although the following embodiments describe that this fluid has typically already been in contact with algae (which caused the depletion of the compound(s) in that fluid) and this provides typically a continuous and efficiently-produced supply of that fluid, it will be appreciated that this need not be the case and that a processing fluid depleted in the compounds desired to be consumed by the algae can be produced in a variety of ways. Typically, the algae are exposed to the fluid in an environment which encourages or enables growth of the algae. This causes the algae to enter a state where the algae will more rapidly consume the compound(s) than they would in a normal metabolic state should they subsequently become present in a fluid to be treated which contacts the algae. This is because when the algae are depleted in the one or more compounds, the operation of different mechanisms within the algae for consuming the compound(s) are up-regulated, increased or transcribed to increase the performance of those mechanisms for consuming those compounds compared to algae in their usual metabolic state. Algae in this state are typically described as “processed”. Processed algae are typically characterised by having a stored or retained concentration or amount of the compound(s) of interest which is lower than the concentration of the compound(s) prior to processing. In some embodiments, the processed algae can be described as either “conditioned” or “pre-conditioned” algae. The processed, conditioned and/or pre-conditioned algae can be added to the fluid in a contact phase in a separate contact receptacle to deplete the compounds in the fluid, with at least a proportion of the algae and/or the algae-contacted fluid being recirculated for further processing, conditioning and/or pre-conditioning. Hence, it can be seen that some embodiments provide an approach which uses distinct, separated, stages which occur for selected residence times under selected conditions for specific purposes which prepare the algae not for cell growth but for the ingestion or internalisation of one or more selected compounds from the fluid to be treated. The processing vessel(s) and the contact vessel(s) are separate vessels with differing contents and which perform different functions in parallel. In particular, the processing vessel receives algae (often recirculated from the separate contact vessel) and a fluid typically depleted in one or more compounds (also often recirculated from the separate contact vessel) in order to produce processed algae. The contact vessel receives a fluid to be treated together with the processed algae typically in order to deplete one or more compounds from the fluid.

Conditioned algae are characterised by typically having a stored or retained concentration or amount of the compound(s) of interest which is lower than the concentration of the compound(s) when the algae is in a normal healthy metabolic state, meaning that normal cell function and dividing may not be possible without an external supply of the depleted compound(s) of interest. In particular, conditioned algae typically have a depleted pool of the compound(s) resulting in a high quota and may even have depleted any extracellular polysaccharides (EPS)-bound compound(s). Also, conditioned algae typically have activated, up-regulated or increased high-affinity transporters/enzymes, in addition to low-affinity cell membrane transporters compared with algae which is not conditioned. Conditioned algae are produced by exposing algae to increased amounts of compound-depleted fluid in an environment which encourages the algae to grow and divide which further depletes the algae's internal stores and further activates the high-affinity transporters in the cell membrane.

Although an optional pre-conditioning process is mentioned below, it will be appreciated that this pre-conditioning process may not be required and instead algae can be conditioned directly, although this may increase the overall energy consumption of the process. As mentioned above, the conditioned algae can then be used in the contact phase to deplete the compounds in the fluid. Various mechanisms for the subsequent consumption of target compound(s) in the fluid by the conditioned algae are described in more detail below.

In some embodiments, a pre-conditioning process may occur which helps to reduce the amount of compound(s) already retained by the algae in a low-energy process where the algae are stored in a dark environment for a period of time. This causes the algae to enter a pre-conditioned state with the different mechanisms within the algae for up-taking or removing the compound(s) being initially up-regulated or their performance increased compared to algae in their normal healthy metabolic state. Pre-conditioned algae are characterised by typically having a stored or retained concentration or amount of the compound(s) of interest which is lower than the concentration of the compound(s) prior to pre-conditioning, but the concentration is still within the normal range for supporting a normal healthy metabolic state meaning that normal cell function and dividing is still possible. In particular, pre-conditioned algae typically have a reduced pool of the compound(s) when compared to algae at the end of a contact phase resulting in a reduced EPS-bound compound(s) compared to algae in their normal healthy metabolic state, but greater amounts of the compound(s) compared to conditioned algae. Also, pre-conditioned algae typically have activated, up-regulated or increased high-affinity cell membrane transporters compared with algae in their normal healthy metabolic state, but less quota for up-take of the compound(s) compared with conditioned algae. Although in some embodiments the pre-conditioned algae are then conditioned, this need not be the case and conditioning may be omitted completely. Instead, the pre-conditioned algae may, as mentioned above, then be used in the contact phase to deplete the compounds in the fluid. In some embodiments, both conditioning and pre-conditioning occurs with both conditioned and pre-conditioned algae being used in the contact phase to deplete the compounds in the fluid.

For those embodiments that perform pre-conditioning and conditioning, subsequently exposing the pre-conditioned algae to larger amounts of the compound-depleted fluid in an environment appropriate for growth in the conditioning process further enhances the performance of the mechanisms. Hence, it can be seen that some of the mechanisms that are upregulated during pre-conditioning are different to those upregulated during conditioning and that additional mechanisms are activated by conditioning. Algae in this pre-conditioned state can be added to the fluid to begin to reduce the concentration of the compound(s), with typically smaller amounts of conditioned algae being added to further reduce the concentration of the compound(s). Performing pre-conditioning helps to reduce the energy consumption of the process. Both the conditioning and pre-conditioning can be performed in the dark. Using conditioned and/or pre-conditioned algae reduces the need for light to take up materials present in the fluid when the conditioned and/or pre-conditioned algae contact that fluid in a contact stage.

Typically, this approach recirculates algae, which become increasingly adapted to the removal of the compound(s) from the fluid. As the algae becomes more adapted, the time taken for the compound(s) to be consumed reduces, which means that the rate at which the fluid can be introduced increases. This is achieved by collecting algae from the conditioning and/or the pre-conditioning stage and exposing that algae to a fluid having the compound(s) to be consumed followed by a portion of time exposing that algae to a recirculated portion of low compound(s) fluid gained at the end of the contact phase. Conditioned and/or pre-conditioned algae used to remove the compound(s) from the fluid, as well as a portion of the fluid following exposure to the conditioned and/or pre-conditioned algae (which is now deplete in the compound(s) is recirculated to the conditioning and/or the pre-conditioning stages. Any excess algae may be harvested with the compound(s) having been retained by that algae for use elsewhere.

Typically, to start the process, stock (unconditioned or untreated) algae can be exposed to the fluid which results in the consumption of the compound(s) at a normal metabolic rate (usually utilising the cell's low-affinity transporters), although this takes longer than when using processed, pre-conditioned and/or conditioned algae (typically days compared with hours for processed, pre-conditioned and/or conditioned algae).

Processing Apparatus

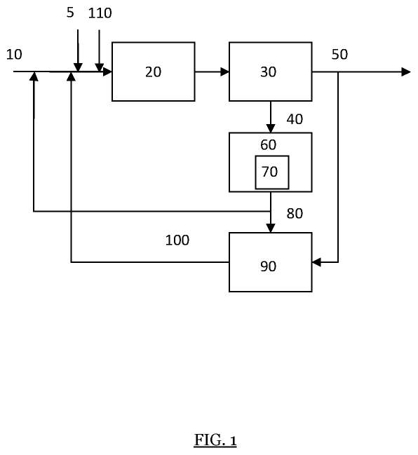

FIG. 1 illustrates schematically an apparatus according to one embodiment. Although this embodiment relates to a continuous flow apparatus, it will be appreciated that a similar approach may be used for batch processing. Some embodiments provide for a continuous or semi-continuous batch process, which provide for the parallel preparation of processed algae, together with the treatment of water or other fluid. The following description describes the operation of the apparatus when consuming or reducing phosphates levels in a fluid, but it will be appreciated that the apparatus can equally be used for other compounds such as nitrogenous, pharmaceutical compounds, metabolites and metallic elements such as zinc and organic compounds. Also, this embodiment describes both pre-conditioning and conditioning algae, as mentioned above, either the pre-condition or the condition can be omitted and instead solely pre-conditioned or solely conditioned algae can be utilised in the contact process.

Initialization

In the absence of any conditioned algae, an initialization process may first be undertaken to provide or create such conditioned algae. It will be appreciated that this process may be omitted if conditioned algae are already available. In this initialization process there is no pre-conditioned or conditioned algae and so algae 5 is supplied with a fluid 10 into the contact vessel 20. The contact vessel 20 may be any suitable vessel which promotes contact between the fluid 10 and the algae 5. For example, the contact vessel 20 may be open or enclosed, such as a pond or a container. During this contact phase, the algae 5 metabolize or otherwise accumulate phosphates present in the fluid 10 using low affinity cell membrane transporters, increasing their biomass and decreasing the concentration of phosphates within the fluid 10 in processes such as growth or cell repair. Initially just the external phosphate (i.e. phosphate in the fluid 10) will be reduced. The cell creates a ‘steady-state’ of internalised phosphate via cell division. The cell phosphate % dry weight will typically remain at around 1-1.5%. The main aim of this initialisation process is to create a low concentration of compound(s) in the fluid 10. Typical up-take rates of phosphates can by around 0.3 mg/L/day. The amount of time that the algae 5 need to be in contact with the fluid 10 in the contact vessel 20 during this initialization process will be longer than subsequently required when using pre-conditioned and conditioned algae (typically days compared with hours for pre-conditioned or conditioned algae). Fluid-exposed algae 40 together with processed, treated or algae-contacted fluid 50 exit the contact vessel 20 when the concentration of the phosphates falls to below a threshold amount. This facilitates the operation of the apparatus since these can be re-circulated. Unlike some existing approaches which recirculate the fluid because there are still nutrients present that can be utilised for cellular growth, some embodiments instead seek retain the fluid-exposed algae 40 together with algae-contacted fluid for a selected residence time, waiting for the absence or reduction in concentration to selected levels of specific nutrients/compounds. The fluid-exposed algae 40 produced by this initialization process will typically have cell phosphate % dry weight of around 1-1.5% whereas recirculated fluid-exposed algae 40 (i.e. conditioned and/or pre-conditioned algae) exiting the contact vessel (discussed below) will typically have cell phosphate % dry weight of around 2.5-16%. In a continuous flow apparatus, this is achieved by controlling the flow of the fluid 10 to achieve an adequate residence time within the contact vessel 20. Typically, a fill and spill/overflow-type system is utilised where one vessel overflows in to the next, this saves power and allows control of retention times. It also prevents short circuiting where fluid or algae put into the front of the process might flow straight though without adequate contact time.

A separator 30 couples downstream with the outflow of the upstream contact vessel 20. The separator 30 separates the fluid-exposed algae 40 from algae-contacted fluid 50. The algae-contacted fluid 50 has reduced levels of phosphates compared to the fluid 10 supplied to the contact vessel 20. The fluid-exposed algae 40 likewise may have increased levels of phosphates compared to the algae 5 supplied to the contact vessel 20 and/or the algae 5 may have grown to provide increased biomass—in either event, the amount of phosphate present in the algae-contacted fluid 50 is reduced compared to that present in the fluid 10. Any excess fluid-exposed algae 40 can wasted or used for other purposes.

Pre-Conditioning

An optional or selective pre-conditioning process is utilized to induce an initial reduction to the biomass phosphate pools, and to some degree to the EPS-bound phosphate, via a low energy passive process. This helps to reduce the time required in a subsequent higher-energy and/or resource intensive conditioning process (for example adding an additional organic carbon source such as glucose). The fluid-exposed algae is provided from the upstream separator 30 to a downstream pre-conditioning vessel 60. The fluid-exposed algae 40 is concentrated by the separator 30 and so has a higher optical density (typically up to 300 times more concentrated) compared to the algae 30 present within the contact vessel 20. Concentrating the fluid-exposed algae 40 reduces the storage volume requirements of the pre-conditioning vessel 60 and reduces the amount of algae-contacted fluid 50 present. The pre-conditioning vessel 60 is unlit and contains an agitator 70 to provide for typically homogenised mixing of the fluid-exposed algae 40 within the pre-conditioning vessel 60. The increase in concentration and the lack of light provides inert conditions for the consumption and attendant reduction of phosphates retained by the fluid-exposed algae 40, with very little external phosphate available within the relatively small amount of algae-contacted fluid 50 present. Typically, this stimulates of production of materials by the algae such as extracellular polysaccharides which are efficient and binding to phosphates and other materials. This is typically due to the algae-contacted fluid 50 being depleted of materials such as phosphates which stimulates the algae to activate mechanisms that are more efficient at catching those phosphates.

FIG. 2 shows the changes in optical density (indicating the change in biomass concentration) and the changes in phosphate retained by the fluid-exposed algae 40 whilst within the pre-conditioning vessel 60. As shown in FIG. 2, the cell phosphate percentage dry weight reduces rapidly during storage within the pre-conditioning vessel 60 from an average of 2.5%+/−0.59% phosphate dry weight to 1.1%+/−0.35% phosphate dry weight after two days of dark storage. As can also be seen in FIG. 2, in some examples, the biomass concentration increases as the cells divide, consuming retained phosphates, with the optical density peaking after three days, which corresponds to the maximum increase in biomass. After three days, the optical density decreases due to an increased mortality of the fluid-exposed algae 40. However, in other examples where no significant cell division occurs, there is no noticeable increase in biomass but a decrease in retained phosphates still occurs (albeit at typically a reduced rate) due to the continued metabolic consumption of retained phosphates. Typically, pre-conditioned algae 80 is produced in a quantity that exceeds steady-state requirements.

Pre-conditioned algae 80 exit the pre-conditioning vessel 60 when the concentration of the retained phosphates falls to below a threshold amount. Typically, this is achieved by retaining the pre-conditioned algae 80 in the pre-conditioning vessel 60 for a selected residence time which has been determined to have caused the retained phosphates to have fallen to below the threshold amount. The pre-conditioned algae 80, while having a reduced retained phosphate level compared to the fluid-exposed algae 40 is still able to perform normal cell functions and divide. In a continuous flow apparatus, this is achieved by controlling the flow of the fluid-exposed algae 40 to achieve an adequate residence time within the pre-conditioning vessel 60. In this example, the fluid-exposed algae 40 will have a residence time of up to around 72 hours within the pre-conditioning vessel 60.

The pre-conditioned algae 80 have typically reduced internal pooled polyphosphate levels, increasing the quota (when compared to the fluid-exposed algae 40) and have started to up-regulate high affinity transporters in the cell membrane in addition to the low affinity transporters creating a pre-conditioned biomass which is able to more quickly take up phosphates in the fluid 10 in a short period of time compared to the fluid-exposed algae 40 and/or algae in their normal healthy metabolic state which consume at their normal or standard range of metabolic rates. Therefore, the pre-conditioned algae 80 can then be recirculated to be used in the contact vessel 20 to boost conditioned algae and induce an initial reduction in phosphate levels in the fluid 10, prior to adding the conditioned algae, as will be described in more detail below. Should the phosphate levels detected in the fluid 10 rise, then up to all of the excess pre-conditioned algae 80 can be recirculated for use in the contact vessel 20.

Hence, it can be seen that the time spent within the pre-conditioning vessel 6o reduces the algae's internalized phosphate pools and has started to up-regulate high affinity transporters in the cell membrane as well as the low affinity transporters under dark conditions which reduces the overall energy consumption of the apparatus.

Conditioning

The pre-conditioned algae 80 supplied by the upstream pre-conditioning vessel 60 are split, with a proportion being directly recirculated back to mix with the fluid 10 entering the contact vessel 20 as mentioned above and the balance being provided to a downstream conditioning vessel 90 and optionally with any excess retained in a holding vessel (not shown). The conditioning vessel 90 receives the pre-conditioned algae 80 and also receives a proportion of the algae-contacted fluid 50 provided by the separator 30. The addition of the algae-contacted fluid 50 dilutes the pre-conditioned algae 80. The conditioning vessel 90 has a highly lit surface area-to-volume ratio. These two changes in conditions enable photosynthesis to occur which encourages the algae to grow and divide. However, the algae-contacted fluid 50 is still depleted in phosphates and so the algae continues to deplete the retained phosphates in its phosphate pools and any EPS-bound phosphate as it repairs chloroplast and utilises lipids during cell division. Typically, the algae are forced to further deplete their internal phosphate stores from around 1% to as little as 0.2% phosphate dry weight during conditioning increasing the potential phosphate quota. The phosphate dry weight can be measured or alternatively algae which show no significant increase in growth for at least three consecutive days may be considered to be phosphate-depleted. Another benefit of using the algae-contacted fluid 50 in the conditioning vessel 90 is that it discourages the growth of other organisms that might compete with or upset the algae.

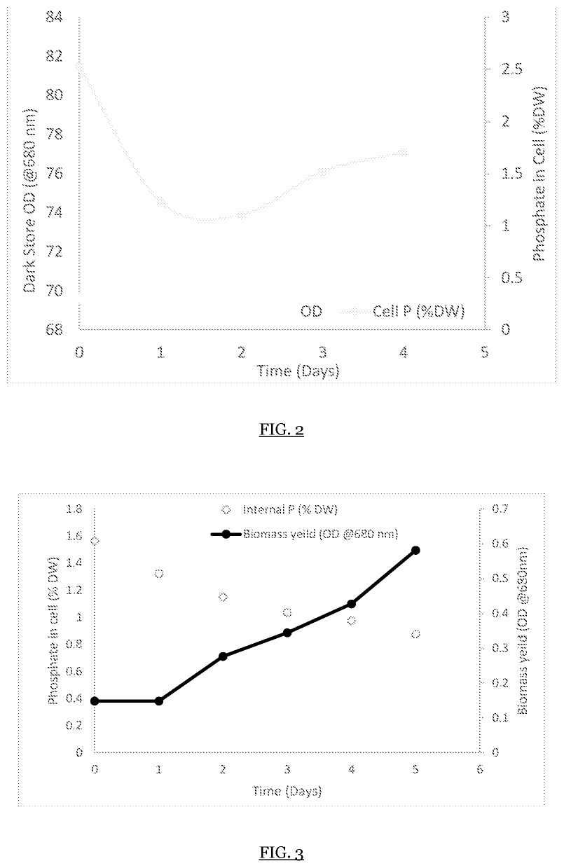

FIG. 3 shows the changes in optical density (indicating the change in biomass concentration) and the changes in phosphate retained by the pre-conditioned algae 80 whilst within the conditioning vessel 90. As can be seen, algae within the conditioning vessel 90 have its phosphate reserves depleted (reducing from around 1.6% phosphate dry weight to around 0.8% phosphate dry weight) while also gaining significant biomass growth (the optical density increases from around 0.15 OD to 0.58 OD). The phosphate level in the conditioned algae 100 is such that the algae are on the edge of not being able to perform normal cell functions and their ability to continue to divide is severely curtailed. In response to the algae reducing the phosphate load, the algae further up-regulates the high affinity transporters in the cell membrane (compared to both the fluid-exposed algae 40 and the pre-conditioned algae 80) to ensure that when the cell next contacts phosphate it can quickly assimilate large quantities and will tend to over-shoot the amount required for normal metabolic functions.

Typically, the pre-conditioned algae 80 are effective at binding to materials in the fluid 10. Conditioned algae 100 are effective at binding to materials in the fluid 10 but can also take them in to the cell much higher rate and quantities, and for longer periods than the algae 5 or the pre-conditioned algae 80.

Although the presence of light is suitable for photoautotrophic algae, for heterotrophic or mixatrophic algae glucose can be added to the conditioning vessel 90 instead of light to provide for conditioned algae 80.

Typically, the conditioning vessel 90 is formed from a series of vessels (set up in a fill and spill/overflow arrangement). This allows the regulation of the retention time and continuous operation. Batches of single tanks could be used, but this would require a more complex arrangement with more valves and pumping. Using one large vessel would typically be more inefficient as it would be more difficult to control the length of time that conditioning occurs for before the biomass is fed on to the contact vessel 20.

Contact

The conditioning vessel 90 outputs conditioned algae 100 which are recirculated for use in the contact vessel 20. The conditioned algae 100 are typically introduced after any pre-conditioned algae 80 and combined with the fluid 10 within the contact vessel 20. The conditioned algae 100 and any pre-conditioned algae 80, together with the fluid 10, are introduced into the contact vessel 20. The fluid 10 is high in phosphate load compared to the algae-contacted fluid 50. The pre-conditioned algae 80 and the conditioned algae 100 have depleted phosphate levels and the up-regulation of the high affinity transporters enable the phosphates present in the fluid 10 to be taken up at a significantly higher rate than by the starter algae 5. Typical up-take rates of phosphates by the pre-conditioned algae 80 and the conditioned algae 100 can by around 0.3-1.9 mg/L/hour. Typically, within six to 16 hours (depending on the starting phosphate load and biomass amount inoculated) the algae within the contact vessel 20 reduce the inorganic phosphate to significantly lower or even undetectable levels and the total phosphate concentration to less than 0.1 milligrams per litre (and often to as low as 0.05 milligrams per litre). At the end of the contact process, the fluid-exposed algae 40 will contain an internal phosphate pool commonly between 1.7 to 4% phosphate dry weight, with up to 16% phosphate dry weight having been measured.

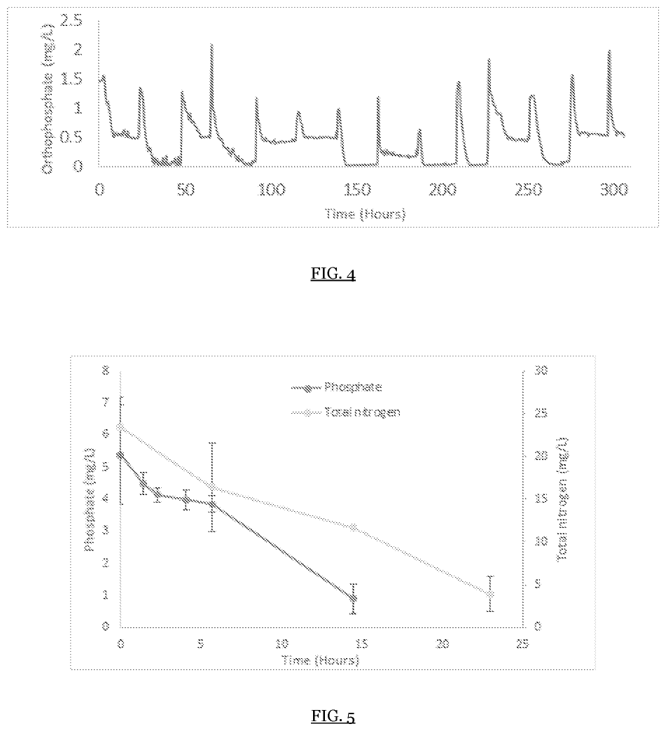

FIG. 4 shows test results from a demonstration facility using batch processing of 25,000 litres of fluid 10 over a two-week period. As can be seen, the phosphate was consistently removed to below 0.5 milligrams per litre.

As mentioned above, in some embodiments only pre-conditioning occurs and only pre-conditioned algae are supplied to the contact vessel 20 and so conditioned algae are omitted in those embodiments. In other embodiments only conditioning occurs and only conditioned algae are supplied to the contact vessel 20 and so pre-conditioned algae are omitted in those embodiments.

FIG. 7 shows the removal of phosphate from wastewater (fluid 10) using just conditioned algae 100 and, alternatively using just pre-conditioned algae 80. The initial phosphate level in the wastewater in the contact vessel 20 was around 5.5 to 6 mg/L. As can be seen, the pre-conditioned algae 80 (having an initial PDW of around 1.2%) is effective for a shorter window of time—with phosphate removal slowing after 4 hours when it reaches around 2 mg/L. However, the removal with only conditioned algae 100 (having an initial PDW of around 0.7%) is consistent and very linear (R2=97%), lasting for 7 hours. Hence, in this example, if it is desired that the phosphate level in the algae contacted fluid 50 leaving the contact vessel 20 is to be around 2 mg/L, then this could be achieved using only pre-conditioned algae 80.

Other Compounds—Combined Phosphate and Nitrogen Removal Through Organic Carbon Reduction

The process can be optimized to reduce other compounds present in the fluid 10. For example, through the addition of processing compounds 110 (for example, an additional organic carbon source such as glucose) the algae within the contact vessel 20 can simultaneously remove total nitrogen and phosphates in less than 24 hours, as illustrated in FIG. 5. This enables the process to be adapted to remove or reduce compounds to meet future needs (for example, to meet future environmental legislation) by combining multiple treatments into a one-stage intensified biological process and releasing algae-contacted fluid 50 that is depleted in both phosphates and nitrogen to the environment, reducing the likelihood of algal blooms and eutrophication. Another key advantage of combined phosphate and nitrogen removal within a microalgae based process is that the nitrogen is captured and chemically bound, whereas in bacteria based treatment processes the nitrogen is reduced to N₂ which escapes to atmosphere.

In some embodiments, the fluid 10 can be a wastewater high in sugar contents, such as sugar cane and brewery wastewater and have high total nitrogen, phosphorus, organic carbon and chemical oxygen demand (COD). In this case the algae in the contact vessel 20 can utilize organic carbon (glucose, acetate, etc.), either in the presence of light (mixotrophic mode) or in its absence (heterotrophic mode) and will grow in the contact vessel 20. The direct availability of organic carbon sources in the form of glucose may avoid the requirement for energy-expensive reactions. Heterotrophic and mixotrophic growth prefers the synthesis of sugar carbon reserves over lipids, resulting in a higher total lipid content and lipid productivity of the algae within the contact vessel 20. The increase in lipid content can be attributed to the increase in cells produced under such conditions, for example heterotrophic and mixotrophically grown algae can result in around a 400% higher biomass yield when compared to phototrophic growth under low organic carbon loads (such as, for example, 20-60 mg/L). Consequently, a higher volume of algae would have to be wasted typically either following separation by the separator 30 and/or following pre-conditioning by the pre-conditioning vessel 70. Furthermore, the presence of an organic carbon source enables the algae in the contact vessel 20 to utilise both ammonium and nitrate, thus enhancing total nitrogen removal, as well as phosphate.

Other Compounds—Heavy Metals

In some embodiments, the fluid 10 can be the discharge of wastewater from industrial, agricultural, municipal, and animal plants which present a significant threat to the environment due to the high risks associated with toxic heavy metals within wastewater effluent. The discharge of such wastewater into the environment threatens public health, should heavy metal concentration exceed the permissible discharge limits for aquatic ecosystems. The concentration of heavy metals, such as Pb²⁺ and Cd²⁺, in municipal wastewater have been reported to exceed discharge standards already greatly. Microalgae offer a distinct advantage for heavy metal remediation given their effective capability of metal uptake. For example, total removal rates, via biosorption and metabolic mechanisms, from mine drainage wastewaters have been reported to be 94.89%, 95.06%, 94.19% and 95% for Fe, Cu, Zn, and Cd, respectively. Microalgal heavy metal removal occurs via two mechanisms (i) a non-metabolic mechanism which involves cell surface adsorption (ion exchange, complexation, and physical adsorption) and (ii) a metabolic dependent uptake mechanism where the heavy metals are actively transported across the membrane in a similar manner to phosphate removal as mentioned above. Therefore, algae in the contact vessel 20 in the presence of excess heavy metals and nutrients can actively uptake and assimilate both contaminants, ultimately eliminating the release of harmful concentrations of toxic pollutants into the environment. Excess algae biomass can then be wasted typically following separation by the separator 30 and used for downstream valorisation, whilst the remaining algae 40 can be pre-conditioned in the now deplete centrate. Both phosphate and heavy metal transporters in the algae will be upregulated in both the pre-conditioning vessel 60 and conditioning vessel 90 in preparation for use in the contact vessel 2030, thus producing a combined process for nutrient and heavy metal bioremediation of wastewater streams.

As mentioned above, metallic elements can also be removed from the fluid 10. Table 1 shows phosphate and zinc levels in the fluid 10 within the contact vessel 20.

TABLE 1

| Time | Phosphate (mg/L) | Zinc (mg/L) | Zinc (% Removed) |
|---|---|---|---|
| 0 | 1.51 | 0.118 | |
| 2 | 1.12 | 0.027 | 77.11 |
| 3 | 0.98 | 0.022 | 81.3 |
| 4 | 0.86 | 0.026 | 77.96 |
| 5 | 0.68 | 0.024 | 79.66 |

The fluid 10 contained 0.118 mg/L of zinc. Without wishing to be bound by theory, it appears the in the algae in the contact vessel instantly bound around 77% of the available zinc, reducing the level to 0.027 mg/L within 2 hours. There appears to be very little change over the next 5 hours. The quick uptake suggests that the metal ions are adsorbed onto different functional groups on the cell. It is expected that the zinc is then absorbed into the cell as the cell does require zinc for metabolic functionality.

Other Compounds—Pharmaceutical and Personal Care Products (PCPs)

In some embodiments, the fluid 10 can be from the aquatic environment. The unintentional presence of pharmaceuticals and personal care products (PCPs) in several compartments of the aquatic environment (e.g., water, sediments, and biota) occurs at concentrations capable of inducing detrimental effects to aquatic organisms. Many of these chemicals can harm ecosystems and their long-term and synergistic effects on human health remain unknown. Algae-based wastewater treatment processes have been shown to be effective in removing PCPs. Various methods such as biotransformation, photo-transformation, algal sorption all contribute to PCP elimination from the water. The relative contribution of each of these processes is currently unknown, however, depending on water chemistry, algae may biotransform PCPs either directly in the contact vessel 30, or indirectly through microbial degradation via the release of exudates present in the centrate within the pre-conditioning vessel 60 and/or the conditioning vessel no. Additionally, the presence of algae has been shown to support indirect photo-transformation. For example, the photo-transformation of the oestrogens 17α-ethynylestradiol and 17β-estradiol over 4 hours increased from 0.4% to 16-37% in the presence of Chlorella vulgaris. Furthermore, triclosan (TCS-antimicrobial and antifungal agent present in PCPs), was shown to undergo photo-transformation and biotransformation in the presence of Scenedesmus. Thus, the combination of illuminated bioreactors as the contact vessel 20 and the conditioning vessel 90, and the presence of bacterial exudates in the centrate would facilitate the removal of toxic PCP and pharmaceuticals.

Other Compounds—Closed Loop Treatment—Vertical Farms and Aquaculture

In some embodiments, the fluid 10 is a hydroponic and/or an aquaculture fluid. Hydroponics is a method of crop production that has been successfully used for the growth of vegetables and flowers. It uses a soilless cultivation method and a nutrient solution dissolved in water which can produce large amounts of wastewater rich in nutrients and organic matters. Another modern farming practice is aquaculture, which is expected to become the main industry providing aquatic products to human beings. However, with the continuous expansion of the scale of aquaculture and the increased production, water pollution has become a serious problem. In both the aquaculture and hydroponics industry pathogens and fungus in the aquatic environment need to be tightly controlled. It has been demonstrated that microalgae cultured under phosphate and nitrogen starvation can display antibacterial and antifungal activity, thereby also reducing pathogens and fungus in the algae-contacted fluid 50. In addition to removing nutrients and reducing bacterial and fungal loads the algae will enrich the dissolved oxygen, which is of benefit to both aquaculture and hydroponics. Harvested waste biomass can also be fed directly back to the aquatic organisms. Therefore, this system can be set up to reuse the algae-contacted fluid 50 with several additional benefits. The residence time in the conditioning vessel 90 and contact vessel 20 can be manipulated to optimise the combination of benefits.

Should there be a significant issue with zooplankton within the fluid 10 (which consume and can decimate algal cultures), such as rotifers, a process that uses an anionic surfactants can be used in the pre-conditioning phase. Both algae and anionic surfactants have a negative charge. Therefore, there is a repelling force between the negatively charged anionic surfactant and the same negatively charged algal cell. Unless damaged and/or dead, the surfactant has little effect of the algal cell. However, the surfactants are able to enter the body cavity of zooplankton through openings, destroying the tissue cells of rotifers. For rotifer eggs, surfactants may cause egg membrane rupture through adsorption and hydrophobicity.

Production Lipids and Pigments

In some embodiments, the apparatus is configured as means of increasing algal products such a pigments or lipid yields within the biomass. Here a designed growth media ideal for algal growth would be supplied as the fluid 10 for maximum biomass yield (slowing down the residence time in the contact vessel 20). Lipid and pigment accumulation in cells would increase in response to incubation at low phosphorus concentration in the conditioning vessel 90 therefore the biomass would be harvested after exiting the conditioning vessel 90 when lipid/pigment stores would be at their highest.

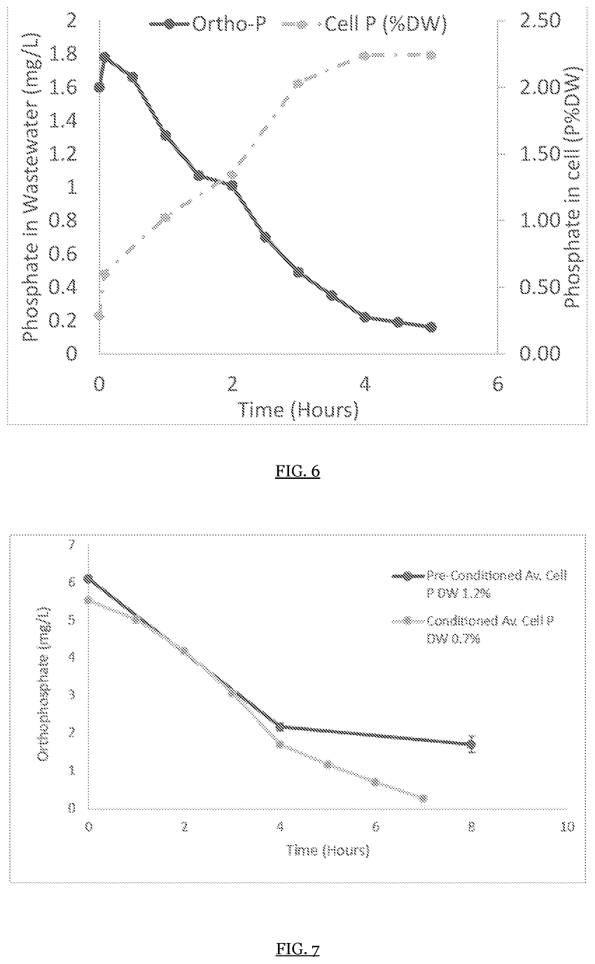

Accordingly, it can be seen that some embodiments provide a phosphate and/or other compound removal and recovery process via the industrial application of microalgae providing a sustainable and environmentally friendly treatment of a fluid or increased yield of products from algal biomass. The process is modular and can be run either in batches or continuously using a modular system which can process the fluid as required (typically to meet current and future environmental legislation). Although fluid processing using microalgae exists, large scale systems typically have a long hydraulic residence time of two to 10 days which constantly supply high loads of phosphate to the algae. This is a relatively unnatural environment for the algae, as the majority of microalgae are naturally acclimated to low phosphate environments. Algae therefore evolve to quickly scavenge large amounts of phosphate in the rate event that it becomes available. Algae respond to phosphate shortages via the up-regulation of high affinity transporters situated in the cytoplasmic membrane of the cell, these transporters efficiently pump inorganic phosphate into the cell in excess of the current metabolic demand for inorganic phosphate. These rapid transporters are typically effective for up to around eight hours once the cell has been re-exposed to phosphate, which can increase the phosphate reserves in the cell from as little as 0.2% phosphate dry weight to as much as 16% phosphate dry weight, as illustrated in FIG. 6. With commonly used approaches, such as high rate algal ponds, the algae are consistently exposed to ample external phosphate. Therefore, low affinity inorganic phosphate transporters are normally expressed within the cell membrane. The expression of the low affinity transporters is characterized by a slower inorganic phosphate take-up rate and expenditure of phosphate for the building of structural components of new cells. Therefore, the removal of phosphate from the fluid is dependent on the consistent and rapid growth of algal cells (typically during an exponential growth phase). If algae are to be applied at any significant scale for fluid processing, that algae will be most efficient if it has been conditioned to upcycle the high affinity inorganic phosphate transporters within the cell membrane. The rapid take-up of phosphate will reduce the required residence time, which is important for reducing the equipment footprint and to treat the range of flow rates entering a treatment apparatus. The apparatus creates the appropriate feast and famine conditions to ensure algae cell readiness for phosphate take-up.

Although illustrative embodiments of the invention have been disclosed in detail herein, with reference to the accompanying drawings, it is understood that the invention is not limited to the precise embodiment and that various changes and modifications can be effected therein by one skilled in the art without departing from the scope of the invention as defined by the appended claims and their equivalents.

1. A method, comprising:
providing processed algae for subsequent use in accelerated consumption of at least one compound from a fluid to be treated by exposing algae to a processing fluid depleted in said at least one compound to be consumed by said algae in a processing vessel;
adding said processed algae to a contact receptacle containing said fluid to be treated to reduce a concentration of said at least one compound from said fluid to be treated; and
recirculating at least a portion of said algae and said processing fluid from said contact receptacle to said processing vessel.

2. The method of claim 1, wherein said processing fluid comprises an algae-contacted fluid which has been in prior contact with algae.

3. The method of claim 1, wherein said exposing said algae to said processing fluid increases a capacity of said processed algae to consume said at least one compound in said fluid to be treated compared to said algae prior to processing.

4. The method of claim 1, wherein said exposing said algae to said processing fluid occurs in conditions which promote a reduction of said at least one compound retained by said processed algae compared to said algae prior to processing.

5. The method of claim 1, comprising recirculating at least a portion of said processed algae by adding said at least said portion of said processed algae to said fluid to be treated.

6. The method of claim 1, wherein said processed algae comprises conditioned algae and said exposing said algae to said processing fluid occurs in conditions which promote a depletion of said at least one compound retained or internalised by said conditioned algae to less than an amount of that compound when said algae is in a normal metabolic state.

7. The method of claim 6, wherein said exposing said algae to said processing fluid occurs in conditions which promote an increase in biomass of said conditioned algae compared to said algae prior to conditioning, and in an illuminated environment.

8. (canceled)

9. The method of claim 6, wherein said method comprises recirculating at least a portion of said conditioned algae by adding said least said portion of said conditioned algae to said fluid to be treated.

10. The method of claim 1, wherein said processed algae comprises pre-conditioned algae and said method comprises pre-conditioning said algae by storing said algae to produce said pre-conditioned algae.

11. The method of claim 10, wherein said storing said algae occurs in conditions which promote an increase in biomass of said pre-conditioned algae compared to said algae prior to pre-conditioning.

12. The method of claim 10, wherein said storing comprises storing said algae for a storage period which is no longer than when a reduction in biomass occurs, and said storing occurs in an illuminated environment.

13. (canceled)

14. The method of claim 10, further comprising:
supplying at least a portion of said pre-conditioned algae to said conditioning vessel;
recirculating at least a portion of said pre-conditioned algae by adding at least said portion of said pre-conditioned algae to said fluid; and
adding at least one of said processed algae, said pre-conditioned algae and said conditioned algae to said fluid to be treated to consume said at least one compound from said fluid to be treated.

15-16. (canceled)

17. The method of claim 10, wherein at least one of said processed algae, said pre-conditioned algae and said conditioned algae consumes said at least one compound from said fluid to be treated at a rate which is faster than algae prior to at least one of said processing, pre-conditioning and conditioning.

18. The method of claim 14, wherein said adding comprises:
adding at least a recirculated portion of at least one of said processed algae, said pre-conditioned algae and said pre-conditioned algae to said fluid to be treated;
adding said pre-conditioned algae to said fluid to be treated prior to adding said conditioned algae;
adding said pre-conditioned algae to reduce an amount of said at least one compound in said fluid to be treated prior to adding said conditioned algae; and
adding said conditioned algae to further reduce an amount of said at least one compound in said fluid to be treated.

19-21. (canceled)

22. The method of claim 10, wherein said compound comprises at least one of a phosphate, nitrogenous, pharmaceutical compounds, metabolites and metallic elements.

23. The method of claim 1, comprising producing said processing contacted fluid by initially adding starter algae to said fluid to be treated and supporting normal metabolic removal of said at least one compound.

24. The method of claim 1, wherein said algae at least partially comprises fluid-exposed algae extracted from said processing fluid.

25. The method of claim 1, wherein said processing fluid at least partially comprises fluid extracted following contact with said algae.

26. The method of any preceding claim 1, wherein said fluid-exposed algae and said processing fluid are extracted by separating at least a portion of said processing fluid.

27. An apparatus, comprising:
a processing vessel configured to provide processed algae for subsequent use in accelerated consumption of at least one compound from a fluid to be treated by exposing algae to a processing fluid;
a contact receptacle configured to add said processed algae to said fluid to be treated to reduce a concentration of said at least one compound from said fluid to be treated; and
a recirculating mechanism configured to recirculate at least a portion of said algae and said processing fluid from said contact receptacle to said processing vessel.
